(19) United States
(12) Patent Application Publication
(10) Pub. No.: US 2022/0160494 A1
(43) Pub. Date: May 26, 2022

(54) Intraocular Device Responsive to Commands

(71) Applicant: Strathspey Crown, LLC, Newport Beach, CA (US)

(72) Inventor: Robert Edward Grant, Laguna Beach, CA (US)

(21) Appl. No.: 17/104,422

(22) Filed: Nov. 25, 2020

(51) Int. Cl. A61F 2/16; A61B 5/15; A61B 5/00; A61B 5/0488; A61B 5/0476

(52) U.S. Cl. CPC A61F 2/1624; A61B 5/150816; A61B 5/0476; A61B 5/0488; A61B 5/742

(57) ABSTRACT

Apparatus and systems that improve or enhance a user's visual experience are provided. The apparatus or system includes an intraocular implant which incorporates a processor, a sensor, and an effector. The apparatus or system is responsive to commands provided by a user, for example spoken commands, gestures, and imagery. All or part of the apparatus or system can be located within the user's eye. Some embodiments include a display, which can provide an enhanced image and/or information related to the apparatus or system.

### FIELD OF THE INVENTION

The field of the invention is intraocular implants, specifically intraocular implants with processing capability.

### BACKGROUND

The following description includes information that may be useful in understanding the present invention. It is not an admission that any of the information provided herein is prior art or relevant to the presently claimed invention, or that any publication specifically or implicitly referenced is prior art.

Intraocular lenses have been used to replace the eye's crystalline lens for over 50 years, typically to restore vision following the development of cataracts. Intraocular lenses in common use can be made from hydrophilic or hydrophobic plastics and typically provide a wearer with a single focal length. Since such lenses lack the ability to provide accommodation, more recently developed intraocular lenses can provide multiple focusing zones and/or provide for a degree of movement of optical elements within the device in order to provide both near and far vision. Even these more advanced lenses are passive devices, however, with limited power for movement of the lens provided by muscles within the eye. In addition, results with these newer designs are mixed.

More recently, intraocular devices have been developed that provide improved accommodation and/or new features. For example, U.S. Pat. No. 10,052,196 (to Pugh et al.) describes an intraocular device that includes a meniscus lens and accessory devices (power sources, processors, sensors, etc.) that provide a user with the ability to adjust the focus of the meniscus lens. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. U.S. Pat. No. 10,123,869 (to Blanckaert et al.) describes an intraocular lens that incorporates a liquid crystal layer, which can adjust the refractive index of the lens in order to change its focus using input from contraction of the ciliary muscles in order to determine the degree of adjustment. U.S. Pat. No. 10,126,569 (to Blum et al.) describes a similar system that utilizes a fixed-focal length lens in combination with electroactive optical elements. The use of information received from the user's brain to control adjustment of similar systems has also been proposed (U.S. Pat. No. 8,778,022, to Blum and Kokonaski).

Such systems and device, however, appear to be limited to adjustment of the focusing ability of an artificial lens. However, such focal adjustment is insufficient to address visual defects caused by many ophthalmic conditions, such as macular degeneration. In addition, such complex active systems and devices (which a user would heavily depend on) lack convenient and unobtrusive means for monitoring and controlling the status of their many components.

Thus, there is still a need for implantable active ophthalmic devices that address the needs of a broad range of ophthalmic conditions.

### SUMMARY OF THE INVENTION

The inventive subject matter provides apparatus and systems that provide an intraocular implant that incorporates an imaging and display technology, and that is responsive to commands provided by a user.

One embodiment of the inventive concept a visual implementation system that is operable by a user, where the system includes a sensor (such as an image detector) that is configured to sense a command, a processor configured to interpret the command and to derive or otherwise generate an output signal from the command, and an intraocular device with an effector that is configured to execute the output signal. Commands can include any action or representation symbolic of an instruction to the processor to generate a specific output signal.

In some embodiments the processor is configured to utilize data from an image detector, and to interpret a visually detectable movement of a body part as a command. In some embodiments the processor is configured to use data from the image detector to interpret a visually detectable text as the command. In some embodiments the processor is configured to utilize data from the image detector to interpret a series of images as the command. In some embodiments the command includes an instruction to alter or modify light that is directed to a retina of the person (e.g. reducing or increasing intensity, modifying frequency bandwidth, shifting a frequency distribution, reducing or enhancing a specified range of frequencies, redirection to a different portion of the retina, etc.)

Various sensors can be utilized. As noted above, in some embodiments the sensor includes an image detector. In some embodiments the sensor includes a sound detector (for example, for receiving a vocalization or spoken word as a command). In some embodiments the sensor includes an electrical signal detector, with data from the electrical signal detector serving as commands. Suitable electrical signals can be obtained from measurement of brain activity

In some embodiments the intraocular device also includes a display. In such embodiments a command can include an instruction to render, using the display, information about a status of the intraocular device. Suitable displays include screen and projection displays. Typical status information can include battery status, memory status, processor status, and security alerts. In some embodiments a command can include instructions to render an image using such a display. Such an image can be realized within the display or projected onto a portion of the eye (e.g. the retina). In such embodiments the command can be to provide an enhanced (e.g. magnified, expanded field of view, contrast enhanced, edge enhanced, etc.) image to the user via the display, for example by altering the focal length of a lens that is included with the intraocular device.

In some embodiments of the inventive concept the intraocular device can include a memory device. In such embodiments a command can include an instruction to alter data in the memory. In some embodiments a command can include an instruction to block deletion of data in the memory, for example protecting images held in a loop memory for designated period of time (e.g. five minutes, ten minutes thirty minutes, etc.). In some embodiments a command can be to initiate or terminate recording of data obtained from a sensor of the device in memory.

A system of the inventive device can include an effector that is configured to provide an electrical signal to the user, for example to a muscle within or coupled to the eye of the person or to the user's brain. In some embodiments the effector can be configured to provide an electrical signal to a non-biological component, for example to a portion of the system that is within the eye of the person.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a cross-section of the human eye.

FIG. 2 is a schematic depiction of a system or device of the inventive concept.

### DETAILED DESCRIPTION

The inventive subject matter provides apparatus and systems that provide an intraocular implant can incorporate a processor, a sensor, and (optionally) a display technology, and that is responsive to commands provided by a user.

One should appreciate that systems and devices of the inventive concept provide an intraocular system that is readily controlled by a user.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. The recitation of ranges of values herein is merely intended to serve as a shorthand method of referring individually to each separate value falling within the range. Unless otherwise indicated herein, each individual value with a range is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Systems and devices of the inventive concept provide a visual implementation system that is operable by a user. A portion or all of such a system or device can be located within an eye of the user. Such a system or device is responsive to commands, which within the context of this application refer to an action, symbol, or other representation that represents an instruction to a processor to perform a specific task or series of tasks. Such a system or device can include a sensor that is suitable for receiving a command, a processor that is communicatively coupled to the sensor, and an intraocular device that includes an effector that executes one or more actions on receipt of an output signal from the processor in response to a command. Systems and devices of the inventive concept can include additional components, such as a display, a memory device, and/or a power source.

Systems and devices of the inventive concept can be constructed of or coated with biocompatible materials. Suitable biocompatible materials include polymers (such as acrylates, methacrylates, silicone polymers, etc.), which can be hydrophilic or hydrophobic. Other suitable biocompatible materials include glasses, ceramics, non-reactive metals (such as gold), and crystalline films (such as sapphire or diamond). Different biocompatible materials can be utilized in the construction of different portions of the device or system. For example, biocompatible polymers can be utilized in the construction of optically active portions whereas ceramics and/or nonreactive metals can be used to encapsulate processors, energy storage devices, and other electronic components.

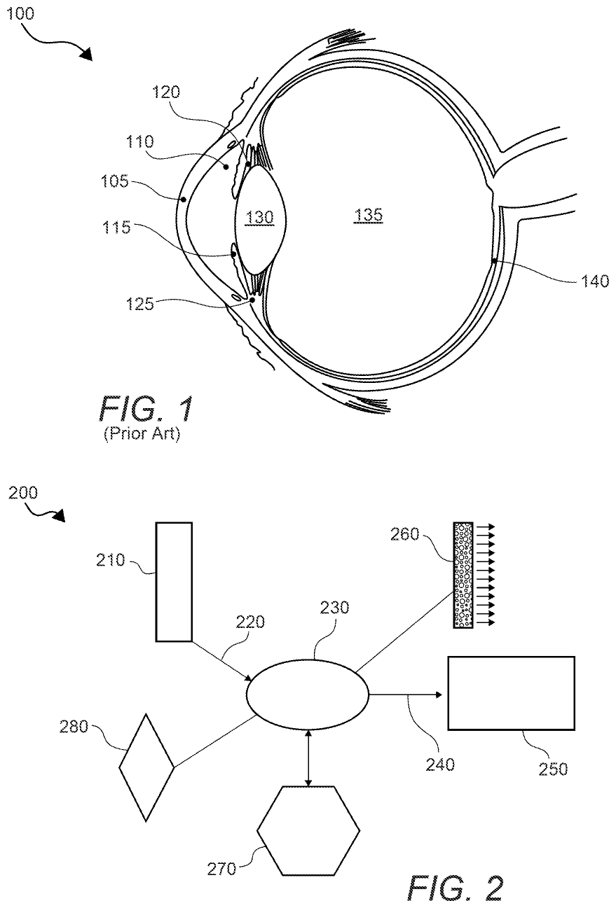

In preferred embodiments, systems and devices of the inventive concept are dimensioned to fit within the confines of a human eye. A cross section of the human eye is provided in FIG. 1. As shown, the human eye (100) includes the cornea (105), which provides a clear outer surface and much of the eye's refractory power, and the light sensitive retina (140) that processes impinging light into nerve impulses that are transmitted to the brain. The anterior chamber or space (110) of the eye lies between the iris (115) and the cornea. The posterior chamber or space (120) of the eye is positioned between the iris and the lens capsule (130). The lens capsule can hold the eye's crystalline lens (not shown), and is also a common site for insertion of an artificial intraocular lens during cataract surgery (following removal of the crystalline lens). The lens capsule is attached to internal structures of the eye by ciliary muscles or bodies (125). The ciliary muscles are used to adjust the shape of the crystalline lens within the lens capsule, and allow the eye to accommodate or shift the plane of focus for the image that is directed to the retina. The vitreous space (135), which is normally filled with a transparent gelatinous material (i.e. vitreous humor), is positioned between the lens capsule and the retina.

All or portions of systems and devices of the inventive concept can be positioned within the anterior chamber of the eye, the posterior chamber of the eye, within the lens capsule, and/or within the vitreous humor. Towards this end components can be constructed of or coated with biocompatible and, where necessary, water resistant materials. For example, electronic components can be encapsulated in sapphire and/or a nonreactive metal. In some embodiments, systems and devices of the inventive concept can be used in conjunction with or incorporate an intraocular lens. In some embodiments placement of the system or device can be accompanied by removal of the crystalline lens of the eye.

Alternatively, in some embodiments of the inventive concept one or more components of the device or system can be located outside of the eye, but remain communicatively coupled to intraocular components. Examples of such externally located components include a processor, memory device, sensor, etc. Such externally located components can be provided on a dedicated device that is worn or otherwise carried by a user (e.g. a pair of eyeglasses, an earpiece, etc.). Alternatively, one or more of such externally located components can be integrated into an existing device that is worn by or otherwise carried by the user (e.g. a smart phone, a smart watch, etc.).

A device or system of the inventive concept is shown schematically in FIG. 2. As noted above, devices and systems of the inventive concept (200) include a sensor (210). Such a sensor is configured and positioned to receive a command provided by a user. Accordingly, the nature of the sensor is at least partially dependent on the nature of the command. In some embodiments the sensor is an image detector, such as a CCD (charge-coupled device) and CMOS (complementary metal-oxide semiconductor) imager. Such an image sensor can be positioned to receive images within or outside of the users normal range of vision. In some embodiments such an image sensor can be provided with accessory optical devices (e.g. a lens, optical aperture, etc.) that facilitate generation of a useful image on the optically sensitive surface of the image sensor. Such an image sensor can be used to receive commands in the form of gestures, written language, specific symbols, or other visual commands. Such an image sensor can be positioned within the eye, or can be positioned external to the eye (for example, coupled to a pair of glasses).

In some embodiments the sensor can be a sound sensor, such as a microphone or similar device. Such a sound sensor can be used to receive commands in the form of spoken language or non-language vocalizations from the user. Such embodiments can include one or more accessory sound sensors, which can be used to monitor background sounds and support background sound reduction. Such a sound sensor can be positioned within or on the surface of the user's eye, or can be positioned external to the eye. In some embodiments such a sound sensor can be incorporated into a portable or wearable device (such as a smart watch or smart telephone) that is typically in the user's possession. In such embodiments the portable or wearable device can include an application that supports one or more functions of the device or system.

In some embodiments the sensor can be an electrical sensor. Such a sensor can be responsive to electrical signals or changes in electric potential associated with actions taken by the user. In some embodiments these electrical signals or changes in electric potential can be a direct output from the brain (e.g. “brainwaves”). In other embodiments such electrical signals or changes in electric potential can be measured from areas outside of the brain, for example as result of muscle activity.

In some embodiments the sensor can be vibration and/or tactile pressure sensor. Suitable sensors include diaphragm sensors, thin film sensors, capacitive sensors, and accelerometers. Such sensors can be activated when the user contacts the sensor, for example by touching a designated sensor. Alternatively, such a sensor can be coupled to the body of the user, and contacted to a convenient surface to activate. In such embodiments the sensor can be coupled directly (for example, as an implant) or indirectly (for example, as an adhesive patch or as part of a garment worn by the user).

It should be appreciated that systems and devices of the inventive concept can incorporate one or more of these sensor types. In embodiments incorporating two or more sensors, output from multiple sensors can be used to designate a specified command. Alternatively, in such embodiments a subset of one or more sensors can be utilized to designate one set of commands and a second subset of sensors utilized to designate a second set of commands.

Such sensors can provide an output signal (220) that is directed to a processor (230), which is configured to recognize the output signal as a command. For example, such a processor can include a pattern recognition algorithm that can interpret data from an optical sensor as a specific gesture or motion of a body part that designates a specified command, text that designates a specified command, and/or a symbol and/or character that designates a specific command. Similarly, such a processor can include a pattern recognition algorithm that can interpret data from an audio sensor as a specific word or other patterned sound that designates a specific command.

On receipt of a command, such a processor is configured to generate a processor output signal (240) to one or more effector(s) (250). In some embodiments the device or system includes a single effector. In other embodiments the device or system includes two or effectors, which can be directed to different purposes. In response to the output signal the effector performs actions that implement the intent of the command. The nature of the effector is dependent on the action required. Suitable effectors include optical devices, piezoelectric devices, artificial muscles, etc. In some embodiments the command can include an instruction to alter or modify light that is directed to a retina of the person. Such modifications include reducing or increasing light intensity, modifying the frequency bandwidth of light entering the eye, shifting frequency distribution of light entering the eye, reducing or enhancing a specified range of frequencies of light entering the eye, redirecting the optical path of light entering the eye (e.g. to a different portion of the retina), and expanding or restricting the field of view directed to a specified portion of the retina.

In some embodiments an effector can be configured to provide an electrical signal to the user, rather than to a component of the device or system. For example, in some embodiments an effector can be configured to provide a signal (such as electrical stimulus) to a muscle within or coupled to the eye of the person. Suitable muscles include muscles of the iris, ciliary muscles, the superior rectus muscle, the inferior oblique muscle, contract together as the inferior rectus muscle, and/or the superior oblique muscle. Such signals can be used, for instance, to control the amount of light entering the eye, adjust the eye's aperture, and/or direct the eye towards an object identified as an object of interest by the controller. In some embodiments such electrical signals can be directed to the user's brain.

In some embodiments an intraocular of the device or system can include a display (260). In such embodiments the display can act as an effector, or can be utilized in addition to or conjunction with an effector. Suitable displays can include passive displays (in which light passes through or reflected from the display) and/or active displays (in which light is emitted from the display). Examples of passive displays include liquid crystal displays and diffusive particle displays (e.g. “e-ink”). Examples of active displays include LED screens, plasma screens, and projector displays. Such a projector display can include a light source (such as an LED or LED laser) that is positioned to direct light towards a directing device (such as a mobile, tiltable, and/or deformable mirror), which in turn can be used to direct projected light toward the user's retina to provide a viewable image.

In such embodiments a command can include an instruction to use such a display to provider a user with information about status of one or more parameters related to the intraocular device. Suitable status information includes, but is not limited to, power consumption, energy generation status, energy storage status, estimated running time on stored power, memory status, processor status, and/or security alerts.

In some embodiments a command can include instructions to use such a display to provide the user with an image. Such an image can be realized as a static or moving image that shows on the surface of or within the display, or can be an image perceived by the user as a result of light projected onto a portion of the eye (e.g. the retina). In such embodiments the command can be to provide an enhanced image that can replace or be overlayed upon an image perceived by the user via normal eye architecture. For example, such a display can provide a magnified portion of the visual field (for example, by altering the focal length of a lens system that is included with the intraocular device), a field of view that expands beyond the user's unaided visual range, contrast enhancement, edge enhancement, and/or motion enhancement. In some embodiments the processor can provide image recognition algorithms to label or otherwise designate specific elements within the user's visual field, for example identifying objects and/or individuals.

Devices and systems of the inventive concept can include a memory device (270). In preferred embodiments such a memory device is incorporated into an intraocular device. Suitable memory can be volatile or nonvolatile, and can range in capacity from 100 MB to 10 terabytes of more. Such memory is communicatively coupled to the processor, and can be used to store algorithms, system-related data, and data obtained from the sensor(s). For example, such a memory can be used to provide a record of charge/discharge cycles for an energy storage device, store system information, and/or store a record of data provided by one or more sensors of the system or device. Such data can be stored until specifically deleted, or can be stored in a memory loop that retains data for a designated period of time. In such embodiments a command can be one or more instructions that alter or access data stored in the memory. For example, a command can include an instruction to block deletion of data stored in the memory, for example to protect images held in a loop memory for designated period of time (e.g. five minutes, ten minutes, fifteen minutes, twenty minutes, thirty minutes, 45 minutes, one hour, two hours, etc.). Alternatively, a command can be to initiate or terminate recording of data obtained from a sensor of the device in memory or to delete data from a designated event or portion of the memory.

Systems and devices of the inventive concept can include a power generation and/or storage system (280) that provide power to the system or device. Although shown in FIG. 2 as connected to the processor (which can be configured to control power distribution to different system or device components), such a power generation or storage system can be independently connected to two or more system or device components. Suitable power generation systems include photovoltaic cells (which can be provided within the eye), thermoelectric generators that utilize the difference between ambient and body temperature, piezoelectric materials that utilize the user's movements or muscle contractions to generate electrical power, and an antenna generates electrical current by induction on exposure to radio frequencies or magnetic fields. Such an antenna can be integrated into structural elements of an intraocular device, such as haptics that hold the device or a portion thereof in position.

Power so generated can range from 0.1 nW to 100 mW or more. If such generation is insufficient to meet peak power requirements of the system or device, generated power can be accumulated and stored in a suitable power storage device. Suitable power storage devices include capacitors and supercapacitor arrays, which can advantageously be fabricated as thin film devices.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A visual implementation system, operable with respect to a person, the system comprising:
a sensor configured to sense a command;
a processor configured to interpret the command, and derive an output signal from the command; and
an intraocular device having an effector configured to execute the output signal.

2. The system of claim 1, wherein the sensor comprises an image detector.

3. The system of claim 2, wherein the processor is configured to utilize data from the image detector to interpret a visually detectable movement of a body part as the command.

4. The system of claim 2, wherein the processor is configured to utilize data from the image detector to interpret a visually detectable text as the command.

5. The system of claim 2, wherein the processor is configured to utilize data from the image detector to interpret a series of images as the command.

6. The system of claim 1, wherein the sensor comprises a sound detector.

7. The system of claim 6, wherein the processor is configured to utilize data from the sound detector to interpret a spoken word as the command.

7. The system of claim 1, wherein the sensor comprises an electrical signal detector.

8. The system of claim 7, wherein the electrical signal detector is configured to detect a brainwave, and the processor is configured to utilize data from the electrical signal detector as the command.

9. The system of claim 7, wherein the electrical signal detector is configured to detect a biosignal other than a brainwave, and the processor is configured to utilize data from the electrical signal detector as the command.

10. The system of claim 1, wherein the sensor comprises a tactile vibration detector.

11. The system of claim 1, wherein the command comprises an instruction to alter light directed to a retina of the person.

12. The system of claim 1, wherein the intraocular device further includes a display, and the command comprises an instruction to render on the display information about a status of the intraocular device.

13. The system of claim 1, wherein the intraocular device further includes a display, and the command comprises an instruction to render an image using the display.

14. The system of claim 1, wherein the sensor comprises an image detector, the intraocular device further includes a display, and the command comprises an instruction to render, the display an enhanced image detected by the image detector.

15. The system of claim 1, wherein the intraocular device further includes a lens, and the command comprises an instruction to alter a focal distance of the lens.

16. The system of claim 1, wherein the intraocular device further comprises a memory.

17. The method of claim 16, wherein the command comprises an instruction to alter data in the memory.

18. The system of claim 16, wherein the command comprises an instruction to block deletion of data in the memory.

19. The system of claim 1, wherein the effector is configured to provide an electrical signal to a muscle in the eye of the person.

20. The system of claim 1, wherein the effector is configured to provide an electrical signal to a non-biological component in the eye of the person.

21. The system of claim 1, wherein the effector is configured to provide an electrical signal to a brain of the person.
